(19) United States
(12) Patent Application Publication
(10) Pub. No.: US 2008/0234662 A1
(43) Pub. Date: Sep. 25, 2008

(54) Constructive Disposal Placed in Artifon Catheter Applied in Perforations Above the Papillae in Fistula-Papillotomy

(76) Inventors: Everson Luiz de Almelda Artifon, Sao Paulo (BR); Alexander Marra Moreira, Sao Paulo (BR); Melchiades da Cunha Neto, Golania (BR)

Correspondence Address: STITES & HARBISON PLLC, 1199 NORTH FAIRFAX STREET, SUITE 900, ALEXANDRIA, VA, 22314, US

(21) Appl. No.: 10/586,614

(22) PCT Filed: Sep. 19, 2005
(86) PCT No.: PCT/BR2005/000190

§ 371 Date: Jul. 19, 2006

(30) Foreign Application Priority Data
Dec. 31, 2004 (BR) PI0406012-1

(51) Int. Cl. A61M 25/098; A61M 25/01; A61B 1/00

(52) U.S. Cl. 604/529; 604/533; 600/104

(57) ABSTRACT

Constructive disposal placed in artifon catheter applied in perforations above the papillae in fistula-papillotomy, where the procedure known as Endoscopic Retrograde Cholangiopancreatography (ERPC) is characterized by the combination of instrumental transpapillary access and the obtaining of contrasting images through pancreatic bile, a result obtained through the use of the artifon catheter device (A) claimed herein, which regards a perforation catheter above the papilla in fistula-papillotomy, to obtain access to biliary passages, making way for an alternative to known surgical techniques, this artifon catheter device (A) presenting a constructive concept composed of a Y connector component (1); a manipulating component of the concentric perforating tube (2); an external concentric tube component (3), within which a concentric perforation tube component (4) is located of which the far end is connected to a needle component (5); radiopaque marks components (6); a manipulating component of the external concentric tube (7); a retraction blockage component (8).

This invention requires the use of the device named “Artifon Catheter”, which is applied in procedures of surgical nature that regard perforations above the papillae in Fistula-papillotomy, with the main objective of obtaining contrasting images through pancreatic biliary.

As predecessors of claimed invention, it is possible to recall the widely diffused concept of procedures used with Endoscopic Retrograde Cholangiopancreatography (ERCP), a characteristic caused by the combination of instrumental transpapillary access, making it possible to obtain contrasting images through pancreatic biliary.

The procedures named canulization are widely known techniques and the first biliary canulization known in writing occurred in 1968 with Mc Cune as the pioneer, but it was Kawai who improved the technical details and the accessories of the medical procedure of canulization.

For a decade, Endoscopic Retrograde Cholangiopancreatography (ERCP) was predominantly of diagnostic nature and contributed in a great proportion with surgical planning.

In 1974, however, Classen-Semling and Kaway performed the first endoscopic papillotomy, thus starting the era of therapeutics in pancreatic bile endoscopy.

Thus, the diagnostic and therapeutic intentions were applied with the same frequency for more than one decade.

At the end of the 1980's and with the intense development of imaging methods, Helical Computerized Tomography and Cholangioresonance came into being and with this the diagnostic aceracea of pancreatic bile illnesses reached the Endoscopic Retrograde Cholangiopancreatography (ERCP). As such, the predominantly therapeutic intention of Cholangioendoscopy is highly recommended highlighting, however, that therapeutic Endoscopic Retrograde Cholangiopancreatography (ERPC) is related to complications due to manipulation and papillary section.

The invention claimed herein can be seen as a treatment similar to the described techniques, with the intention of performing endoscopic procedures over the papilla, causing the least proportion of trauma, thus creating the concept of microtraumatic “papillotomy”.

In microtraumatic “papillotomy” there is no papillary section, neither any dissipated electric current but a perforation above the papilla is made, characterized by the fistula-papillotomy through perforation

The procedure referred to allows for deep biliary access, making use of a guiding line, pancreatic bile dilatations, passage of prosthesis and finally taking out calculus up to six millimeters (6 mm).

The procedure, based on the technique of a perforation above the papilla, is performed on patients forwarded to the digestive endoscopy sector, who present a clinical profile with the indication of endoscopic cholangiography.

The patients referred to are kept in hospital for 24 hours, the minimum amount of time necessary to obtain the laboratory profile trough serial doses of amylase, pypase, RCP and interleucyne-6, before and after 4, 12 and 24 hours of the procedure.

Clinical analysis is performed by endoscopists and the presence of abdominal pains is verified in bandages, nausea and vomit to characterize the occurrence of acute pancreatitis.

In the event of complications, which are limited, specific measures for each case shall be taken.

In case of Acute Pancreatitis, the procedure adopted are hospitalization, fasting, hydroelectric restitution, tomographic evaluation and evaluation of the graveness using Ransons's criteria.

In case of extensive Submucosa Hematoma after the perforation, the procedure adopted is based on fasting, laboratory and ultrasonic characterization of biliary obstruction.

May other complications occur, these will be taken care of appropriately through diverse procedures.

The main objective involved resides in the evaluation of technical and laboratory profiles of the fistula-papillotomy through perforation above the papilla with subsequent treatment of stent implants by means of endoscopy and/or dilatation with balloons.

Therefore, the use of the Artifon Catheter has the objective to perforate and to create access above the papilla by means of the fistula-papillotomy to view the biliary passages.

To make the it possible to reach the objective referred to above, a device was developed with a constructive concept based on a product of the catheter type, the latter composed of two concentric tube elements that differ in diameter, in which the larger concentric tube has the function of being a guide to the smaller one, which is called concentric perforator tube, and the concentric guide tube possesses greater internal luminosity which makes it possible for the concentric perforator tube to slide within it.

The internal tube, or concentric perforator tube, is used to perforate, a function that is performed by a needle fastened to it, and shall therefore have an internal diameter that makes it possible for a guiding line (with measures that comply with the procedure) to pass.

Also foreseen is the injection of the contrast through the internal luminosity of the concentric perforation tube.

A component of the Y connector type is connected to its extreme end and the injection of the contrast with a guiding line inserted within it.

The functional concept of the product of the catheter type has two opposing steps, the first one corresponding to the non-exposure of the needle.

In this position the needle component is located within the concentric guide tube.

In the second step, the needle component is exposed, a position ready for the perforation operation.

The constructive concept of the product of the catheter type also foresees a blocking mechanism between the aforementioned concentric tubes with the function of avoiding the concentric perforating tube to return during the perforating operation, the blocking operation being performed by means of male-female connector elements.

The set is used together with the endoscope device, inserted through the x canal of the endoscope.

The length of the catheter product is sufficiently longer that that of the endoscope, making external manipulation possible of the catheter product and the extreme free length for the perforating function.

To obtain a total and complete view of how the “Artifon” catheter product in question, which is the object of the claim of this invention patent, is constituted, illustrative drawings are attached, which do by no means limit the preferable performance of the invention, and to which reference is made as follows:

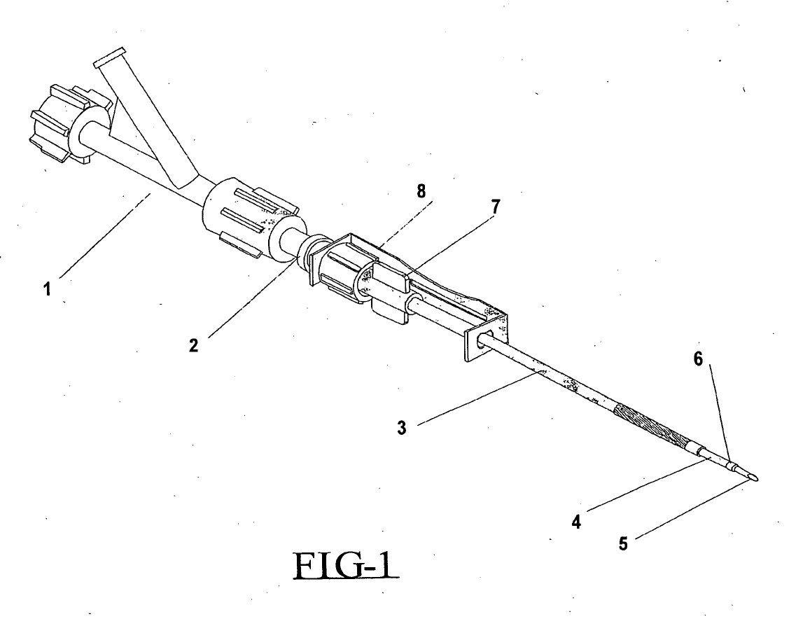

FIG. 1 is a representation in perspective view of the artifon catheter device being claimed.

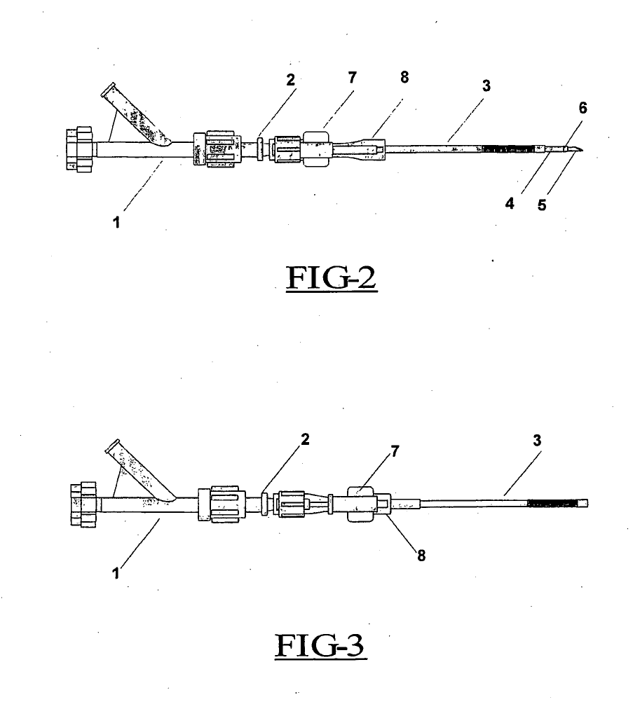

FIG. 2 represents a side view of the artifon catheter device being claimed, with the needle component exposed.

FIG. 3 represents a side view of the artifon catheter device being claimed, with the needle component in the resting position.

With reference to the illustrations, this invention patent requests refers to an ARTIFON CATHETER APPLIED IN PERFORATION PROCEDURES ABOVE THE PAPILLA IN FISTULAPAPILLOTOMY, which is represented in FIG. 1 with alphabetic reference (A), a graphical representation that shows the claim solution, which has a constructive concept that is based com the Y connecting component (1), which has the function of promoting the injection of the contrast, even with the guide line element inserted within its internal luminosity.

The artifon catheter device (A) is used together with a device of the endoscope type, inserted through the x canal of the latter, where its length shall be sufficiently longer than the endoscope itself to provide for external manipulation and free length at the extreme ends for the perforator.

The set formed by the artifon catheter (A) has an external diameter that is smaller than 8 F (French) and is compatible with guideline 0.035″.

A manipulating component of the concentric perforator (2) is foreseen, defined as a part that is fixed to the near extremity of the concentric perforator tube component (4), with the function of manipulating this tube.

Preferably, the manipulating component of the concentric perforating tube (2) has the form of male, female or male/female connectors with standard connections, made in thermoplastic polymers.

Beside the manipulating function of the concentric perforating tube component (4), the manipulator component of the concentric perforation tube (2) presents a secondary function which is allowing for the blockage of the exposure or retention of the needle component (5).

The concentric perforation tube component (4), preferably manufactured in PTFE, is conducted by the inner side of the external concentric tube component (3), also preferably manufactured in PTFE, composed of material that has properties that facilitate sliding of the concentric perforating tube component (4) within it, being able to support sharp bends without breakage or damage along its extension.

If necessary, for the regions where critical bends are present, the external concentric tube component (3) can present reinforcement through other material, which can be in the form of metal or polymer meshes, placed at its extreme end, while it is mandatory to present free passage, that is no restrictions to the concentric perforation tube component (4).

The kinematics of the mechanism of the artifon catheter device (A) claimed, is based on the sliding of the concentric perforation tube component (4) inside the luminosity of the external concentric tube component (3), by this means enabling the perforating operation.

At its turn, the needle component (5), preferably manufactured in steel, is fastened at the far end of the concentric perforation tube (4), while its profile is equal to that of a needle for a perforation operation.

It is desirable that the needle component (5) be made of material of sliding properties with a certain rigidity to avoid it be excessively shortened during the perforation, while, complementarily, it must be possible to make sharp bends, because it accompanies the path of the concentric perforation tube component (4), and it shall also have an internal diameter that makes the passage of a guide line of due size possible.

Also foreseen is the presence of radiopaque marks (6), made in gold, attached to the needle component (5) to view the far end in x-ray, this mark being made in biocompatible radiopaque material.

At its turn, the manipulating component of the external concentric tube (7) is formed by a part that is attached to the near end of the external concentric tube component (3), with the objective of manipulating this tube.

Together with the manipulator of the concentric perforation tube (2), it allows for the blockage of the exposure mechanism or the retention of the needle component (5).

Finally, also foreseen is a retraction blockage component (8), with a blockage function date restricts the retracting movement of the concentric perforator tube component (4).

In relation to its functional concept, the artifon catheter device (A) is characterized for presenting a mechanism that uses opposing kinematics between two concentric perforator tube components (4).

The first position corresponds to that of non-exposure of the needle component (5), represented through FIG. 2, in that the needle is placed in the inner part of the external concentric tube component (3).

At its turn, the second position corresponds to the exposure of the needle component (5), represented by FIG. 3, where it is exposed and ready for the perforating operation.

To avoid the return of the concentric perforating tube component (4) the retraction blockage component (8) is triggered, avoiding the returning movement of the concentric perforating tube component (4), inside the external concentric tube component (3), when performing the perforating operation.

At its turn, the contrast injection occurs by mans of a guideline element, that is inserted within the concentric perforating tube component (4). By this means, it is necessary to use an accessory that allows for this type of injection, where Y (1) connector components are used, which are duly connected through manipulator components of the concentric perforation tube (2).

Essentially, the perforating operation that makes use of the artifon catheter device (A), can occur through the sliding of the concentric perforation tube component (4) within the external concentric tube component (3) or, alternatively, when the artifon catheter device (A) is in the blocking condition, where the operator manually uses the artifon catheter device (A) on the surface to be perforated.

Through everything that is described and illustrated, this writing regards a unique solution en ARTIFON CATHETER APPLIED IN PROCEDURES FOR PERFORATION ABOVE THE PAPILLA IN FISTULA-PAPILLOTOMY, complying with the norms that dictate invention patents, deserving, by means of what has been exposed and as a consequence, respective privilege where, to compose the considerations contained in this writing, the following titles were used as for reference:
1—McCune W S, Shorb P E, Moscovitz H. Endoscopic cannulation of the ampulla of Vater: a preliminary report. Ann Surg 1968; 167(5): 752-6.
2—Kawai K, Akasaba Y, Murakami K, et al. Endoscopic Sphincterotomy of the ampulla of Vater. Gastrointest Endosc 1974; 20(4): 148-51.
3—Demling L, Koch H, Classem M, et al. [Endoscopic papillotomy and removal of gallstones: animal experiments and first clinical results]. Dtsch Med Wochenschr 1974; 99 (45): 2255-7.
4—Freeman M L. Adverse outcomes of endoscopic retrograde cholangiopancreatography: avoidance and management. Gastrointest Endosc Clin N Am 2003; 13 (4): 775-98.
5—Vandervoort J, Soetikno R M, Tham T C, et al. Risk factors for complications after performance of ERCP. Gastrointest Endosc 2002; 56(5): 652-6.

1-8. (canceled)

9. An artifon catheter comprising:
(a) a concentric perforating tube attached to a manipulation component on a first extremity and to a needle on a second opposite extremity;
(b) a radiopaque mark component externally attached to the needle;
(c) an external concentric tube having a first extremity attached to the manipulation component of the perforation tube and a second opposite extremity; internally bearing the concentric perforation tube, the needle and the radiopaque mark component, and having an external manipulating component adjacent to the manipulation component of the perforation tube;
(d) a retraction blockage component externally attached to the external concentric tube portion, and
(e) an Y-shaped connector linearly attached to the manipulating component of the perforating tube.

10. An artifon catheter according to claim 1, wherein the concentric perforation tube and the needle have internal diameters of a size sufficient to enable a guiding line of a due measure in regards to the perforation procedure, to pass through it.

11. An artifon catheter according to claim 1, wherein the manipulation component of the perforation tube is a male-female connector with standard connections.

12. An artifon catheter according to claim 1, wherein the manipulation component of the perforation tube is manufactured in thermoplastic polymer.

13. An artifon catheter according to claim 1, wherein the external concentric tube is manufactured in a composed material facilitating the sliding of the perforation tube through it.

14. An artifon catheter according to claim 1, wherein the external concentric tube portion further presents reinforcements selected from a group consisting of metal of polymer meshes, spiral metal wires and combination of both.

15. An artifon catheter according to claim 1, wherein the reinforcements are placed on the first and second opposite extremity.

16. An artifon catheter according to claim 1, wherein the external concentric tube portion is manufactured in Polytetrafluoroethylene (PTFE).

17. An artifon catheter according to claim 1, wherein the needle presents a rigidity enabling sharp bends.

18. An artifon catheter according to claim 1, wherein the needle is manufactured in steel.

19. An artifon catheter according to claim 1, wherein the radiopaque mark component is manufactured in a biocompatible radiopaque material.

20. An artifon catheter according to claim 1, used together with an endoscope device.

21. An artifon catheter according to claim 1, wherein the radiopaque mark component is manufactured in gold.

22. A method of using an artifon catheter according to claim 1, the method comprising the steps of:
(i) placing the catheter on the surface of a target
(ii) sliding the perforating tube and the needle within the external concentric tube portion generating a perforation operation on a surface of the target;
(iii) access the papilla of a target patient through fistula-papillotomy, and
(iv) viewing the biliary passages of the target.

23. A method of using an artifon catheter according to claim 14, wherein alternatively in steps (i) and (ii) the generating a perforation operation is performed by activating the retracting blockage component, placing the catheter on the surface of the target, and performing a perforation manually.

24. A method of using an artifon catheter according to claim 14, further comprising the steps of:
(v) attaching a Y-shaped connector attached to the manipulating component of the perforating tube;
(vi) injecting a contrast through the guiding line inserted in the internal diameter of the perforating tube.
